(19) United States
(12) Patent Application Publication
(10) Pub. No.: US 2009/0291486 A1
(43) Pub. Date: Nov. 26, 2009

(54) ORGAN PRESERVATION APPARATUS AND METHODS

(75) Inventor: Marshall S. Wenrich, Plano, TX (US)

Correspondence Address: MCANDREWS HELD & MALLOY, LTD, 500 WEST MADISON STREET, SUITE 3400, CHICAGO, IL, 60661, US

(73) Assignee: ORGAN TRANSPORT SYSTEMS, INC., Houston, TX (US)

(21) Appl. No.: 12/535,103

(22) Filed: Aug. 4, 2009

Related U.S. Application Data
(63) Continuation of application No. 10/756,169, filed on Jan. 13, 2004, now abandoned.

(51) Int. Cl. C12M 3/00

(52) U.S. Cl. 435/284.1

(57) ABSTRACT

A transportable organ preservation system that substantially increases the time during which the organ can be maintained viable for successful implantation into a recipient is disclosed. A chilled oxygenated nutrient solution can be pumped through the vascular bed of the organ after excision of the organ from the donor and during transport. The device of the present invention uses flexible permeable tubing to oxygenate the perfusion fluid while the CO₂ produced by the organ diffuses out of the perfusion fluid. One pressurized two-liter “C” cylinder can supply oxygen for up to 34 hours of perfusion time. The device can use a simple electric pump driven by a storage battery to circulate the perfusion fluid through the organ being transported. The vessel containing the organ to be transported can be held at a suitable temperature by a chiller.

### CROSS-REFERENCE TO RELATED APPLICATIONS

This application is a continuation of U.S. Ser. No. 10/756,169, filed Jan. 13, 2004, now pending.

The subject matter of this application is related to U.S. Pat. No. 6,677,150 and to the application identified as Attorney Docket No. 13241US03, filed Jan. 13, 2004, by Samuel D. Prien, All of each application or patent identified in this specification is incorporated here by reference in its entirety.

### STATEMENT REGARDING FEDERALLY SPONSORED RESEARCH OR DEVELOPMENT

Not applicable.

### FIELD OF THE INVENTION

This invention relates to a mammalian organ preservation system, and more particularly to a preservation system that substantially increases the time during which the organ can be kept viable for successful implantation into a human or other mammal recipient. One embodiment of the invention is a transportable system, useful when the organ is excised from a donor at one location and transplanted to a recipient at a different location. A chilled oxygenated nutrient solution can be pumped through the vascular bed of the organ after excision of the organ from the donor and during transport.

### BACKGROUND OF THE INVENTION

Organs have been successfully transplanted since 1960, owing to the improvement of surgical techniques, the introduction of by-pass circulation and the development of drugs that suppress immune rejection of the donor organ. At the present time, the donor organ is harvested under sterile conditions, cooled to about 4° C., and placed in a plastic bag submerged in a buffered salt solution containing nutrients. The solution is not oxygenated and is not perfused through the organ blood vessels. If the organ is to be transported to a recipient or held for a period (instead of being used immediately), the plastic bag is placed in a picnic cooler on ice, transported to the recipient, and finally implanted into the recipient.

For the forty-year history of organ transplantation surgery, maintaining the quality and viability of the organ has been an enormous challenge. The need is great for a truly portable device that nurtures and oxygenates the organ throughout the entire ex-vivo transport.

Currently, when a heart, lung, liver, or certain other organs are harvested from a donor, medical teams have about four hours to transplant the harvested organ into the recipient. Damage to all the organs at the cellular level occurs even during this short period. The current method of transport, called topical hypothermia (chilling the organ in a cooler), leaves 12% of organs unusable because of their deteriorated physiological condition. Thousands of people die each year while on an ever-expanding waiting list.

The lack of donor organ availability, particularly hearts, lungs, and livers, is a limiting factor for the number of organ transplants that can be performed. At the present time, less than 25% of patients who require a heart transplant receive a new heart, and less than 10% of patients who require a lung transplant receive one. A major consideration is the length of time that a donor organ will remain viable after it is harvested until the transplant surgery is completed. The donor organ must be harvested, transported to the recipient, and the transplant surgery completed within this time limit. Thus, donor organs can be used only if they can be harvested at a site close to the location where the transplant surgery will take place.

It has long been known that organs will survive ex vivo for a longer time if they are cooled to a temperature near freezing, typically 4° C., and actively perfused through their vascular beds with a buffered salt solution containing nutrients, and that ex vivo survival of an isolated organ can be further extended if the solution is oxygenated. Several factors play a role in the prolonged survival. At 4° C. the metabolism is greatly reduced, lowering the requirements for nutrients and oxygen, and the production of lactic acid and other toxic end products of metabolism are also greatly reduced. Circulation of the perfusion fluid replenishes the oxygen and nutrients available to the tissue, and removes the lactic acid and other toxic metabolites. The buffered solution maintains the pH and tonic strength of the tissue close to physiological.

Perfusion that allows the transport of a harvested organ from a site removed from the location where the transplant surgery will be carried out requires the use of a lightweight portable device for pumping the cold buffered nutrient salt solution through the organ blood vessels, and in which the organ also can be transported from the site of harvest to the site of implantation. For one person to carry the entire assembly without assistance, and to transport it in an auto or airplane, it desirably would be compact, sturdy and lightweight. The system for loading the perfusion fluid desirably would be simple and allow minimal spillage. The system desirably would oxygenate the perfusion fluid. The device desirably would have a pump with a variably adjustable pumping rate, which pumps at a steady rate once adjusted. Sterility must be maintained. To be completely portable, the device desirably would contain a source of oxygen, an energy source to operate the pump, and desirably would be housed in an insulated watertight container that can be kept cool easily. An entirely satisfactory device has not been available.

U.S. Pat. No. 5,965,433 describes a portable organ perfusion/oxygenation module that was said to employ mechanically linked dual pumps and mechanically actuated flow control for pulsatile cycling of oxygenated perfusate. That patent contains an excellent description of the state of the art in the mid-nineties and the problems associated with transport systems for human organs.

Hypothermic, oxygenated perfusion devices are known in the art and have proven successful in maintaining viability of a human heart in laboratory settings. While different devices are available for laboratory use under constant supervision, none are truly independently functioning and portable.

For example, Gardetto et al., U.S. Pat. No. 5,965,433 describes an oxygen-driven dual pump system with a claimed operating capacity of 24 hours using a single a 250-liter oxygen bottle. The intent of this device was to provide a user-friendly device that would be “hands off” after the organ was placed in the unit. Four major problems were evident. (1) The unit contains no bubble trap and removing bubbles is difficult and time consuming. (2) The lubricant in the pumps dries out after 10 or fewer hours of operation and the pumps stop. (3) At lower atmospheric pressure such as in an aircraft in flight, the pump cycles rapidly due to the reduced resistance to pumping, risking the development of edema in the perfused organ; and (4) Two bottles of oxygen failed to produce more than 16 hours of steady operation.

Doerig U.S. Pat. No. 3,914,954 describes an electrically driven apparatus in which the perfusate is exposed to the atmosphere, breaking the sterility barrier. It must be operated upright, consumes oxygen at high rates, and is heavy. The requirement for electric power and the necessity for a portable source of electric power severely limit the portability of this unit.

O'Dell et al., U.S. Pat. Nos. 5,362,622; 5,385,821; and 5,356,771 describe an organ perfusion system using a fluidic logic device or a gas pressure driven ventilator to cyclically deliver gas to a sealed chamber connected to the top of the organ canister. Cyclical delivery of gas under pressure to the upper sealed chamber serves to displace a semi-permeable membrane mounted between the gas chamber and the organ canister. Cyclical membrane displacement acts to transduce the gas pressure into fluid displacement on the opposite side, providing a flow of the perfusing solution.

The membrane is chosen for its permeability to gas but not to water. This permits oxygen to flow through the membrane to oxygenate the fluid and vent carbon dioxide from the fluid. The intent of such devices is to provide a system that uses no electricity, uses low gas pressure to achieve perfusate flow, has few moving parts, provides oxygenation of the fluid, can be operated in a non-upright position, isolates the organ and perfusate from the atmosphere, is of compact size and low weight to be portable.

These systems fail to meet criteria claimed by the developers. For example, the amount of oxygen necessary to cycle the membrane is very large. When calculated over a 24-hour period, it would require 4 large tanks of oxygen to assure continuous operation. This amount of oxygen fails to meet the definition of portable. The pressure and volume of oxygen required to cycle the membrane is sufficient to cause tearing of the membrane or displace it from its margins. Either of these occurrences would be catastrophic to the organ. The manner in which fluid is cycled into the organ chamber attempts to perfuse both within and around the organ, providing freshly oxygenated fluid to infiltrate and surround the organ. This procedure is without physiological basis since, oxygenation is normally achieved by oxygen diffusion outward from the organ's vascular bed.

All of these devices use a permeable membrane permeable to gas but not to water, with the intention that oxygen or other gas mixtures can be driven through the membrane into the perfusate and can vent the CO₂ produced by the organ, from the perfusate.

The successful use of permeable membranes that are subjected to repetitive variations in pressure over long periods of time depends upon the membrane having elastomeric properties to withstand such repeated flexing without tearing or rupturing. Gas permeable membranes have not been made having such elastomeric properties.

### BRIEF SUMMARY OF THE INVENTION

One aspect of the present invention is apparatus including a perfusion fluid loop for maintaining an ex vivo organ in viable condition for transplantation. The perfusion fluid loop includes an organ container, a bubble remover, and an oxygenator. The organ container receives an organ to be transported. The bubble remover removes gas bubbles from perfusion fluid circulating in the perfusion fluid loop. The oxygenator supplies oxygen to and removes carbon dioxide from perfusion fluid circulating in the perfusion fluid loop.

Another aspect of the invention is an organ transporter for containing, supporting, and perfusing an ex vivo organ. The organ transporter includes an organ container as described previously, defining an organ chamber, and an adapter. The adapter has a first portion defining a hose connector and a second portion adapted for connection to a vessel of an organ in the organ chamber for directing a perfusion fluid into the vessel.

Yet another aspect of the invention is a perfusion fluid comprising a free radical scavenger in an amount effective to increase the length of the period during which the ex vivo organ will remain viable in the perfusion fluid.

Still another aspect of the invention is a composition comprising a free radical scavenger in time-release form adapted for releasing the scavenger into a perfusion fluid over a period of time. For example, the time-release composition can be particles of an organosiloxane material in which a free radical scavenger is dispersed.

The present invention optionally provides a method and apparatus which allows one pressurized two liter “C” cylinder that contains 255 liters of oxygen at standard temperature and pressure to supply up to 34 hours of perfusion time and uses a simple electric pump driven by a storage battery to circulate the perfusion fluid through the organ being transported.

The present invention is contemplated to significantly diminish the problem of limited transport time by providing an apparatus that will extend the transport time to up to 48 hours. This increased time will inherently increase the size of the donor pool and will allow for extensive disease testing and matching.

The present invention is contemplated to reduce damage to the organ being transported and allow organs from post-mortem donors to be used. Today, organs are only harvested from donors who are brain-dead but whose organs have never ceased to function.

Particular advantages of the transport system of an embodiment of the present invention are that it can be easily loaded and unloaded by double-gloved surgical personnel and that the fittings require minimal dexterity to assemble and disassemble.

Another advantage of an embodiment of the present invention is that the device can be devoid of flat membranes and instead can use flexible permeable tubing to oxygenate the perfusion fluid while the CO₂ produced by the organ diffuses out of the perfusion fluid. Flexible flat permeable membrane of the prior art, due to their constant flexing when used as diaphragms for pumping, are subject to fatigue stresses and rupture with catastrophic results.

The use of an embodiment that is lightweight, cooled, self-contained, and provides perfusion is contemplated to have one or more of the following beneficial consequences. (1) The organs will be in better physiological condition at the time of transplantation. (2) Prolonging the survival time of donor organs will enlarge the pool of available organs by allowing organs to be harvested at a greater distance from the site of the transplant surgery in spite of the attending longer transport time. (3) It will allow more time for testing to rule out infection of the donor, for example with AIDS, hepatitis-C, herpes, or other viral or bacterial diseases. (4) The pressure on transplant surgeons to complete the transplant procedure within a short time frame will be eased. Transplant surgeons are often faced with unexpected surgical complications that prolong the time of surgery. (5) Better preservation of the integrity of the organ and the endothelium of the arteries at the time of transplantation is contemplated to lessen the incidence and severity of post-transplantation coronary artery disease.

In one embodiment, the components, and in particular the components that come into contact with sterile perfusion fluid, can be made by injection molding.

### BRIEF DESCRIPTION OF THE SEVERAL VIEWS OF THE DRAWING

The advantages and features of the invention described herein can be understood in more detail by reference to the following description and drawings appended hereto and which form part of this specification.

The appended drawings provide illustrative embodiments of the invention and are therefore not to be considered limiting of its scope.

FIG. 1 is a hydraulic circuit diagram showing the interconnection of the principal components of a portable perfusion apparatus of one embodiment.

FIG. 2 is an expanded perspective view of the embodiment of FIG. 1.

FIG. 3 is a plan view of the apparatus of FIG. 1.

FIG. 4 is a cross-section view of the apparatus of FIG. 1 taken along the lines 1A-1A of FIG. 3.

FIG. 5 is a cross-section view of the apparatus of FIG. 1 taken along the lines 1B-1B of FIG. 3.

FIG. 5a is a detailed view of the lid-container sealing arrangement of FIG. 1.

FIG. 5b is a perspective view of the adapter of FIG. 1.

FIG. 6 is a side view of the apparatus of FIG. 1.

FIG. 7 is a schematic detail view of one embodiment of a cooling pack with a built in heat exchange coil for cooling the perfusion fluid.

FIG. 8 is a schematic view of an alternative arrangement of the components in the organ transporter, with the cooling pack located below and in thermal contact with the organ container 8.

FIG. 9 is a general schematic view of an alternative embodiment of the organ transporter, showing representative elements that can be single-use elements versus multiple use elements of the organ transporter.

FIG. 10 is a more detailed schematic view of an embodiment of the perfusion loop and its control system.

FIG. 11 is a schematic detail view of an embodiment of the electrical power supply of the organ transporter.

### DETAILED DESCRIPTION OF THE INVENTION

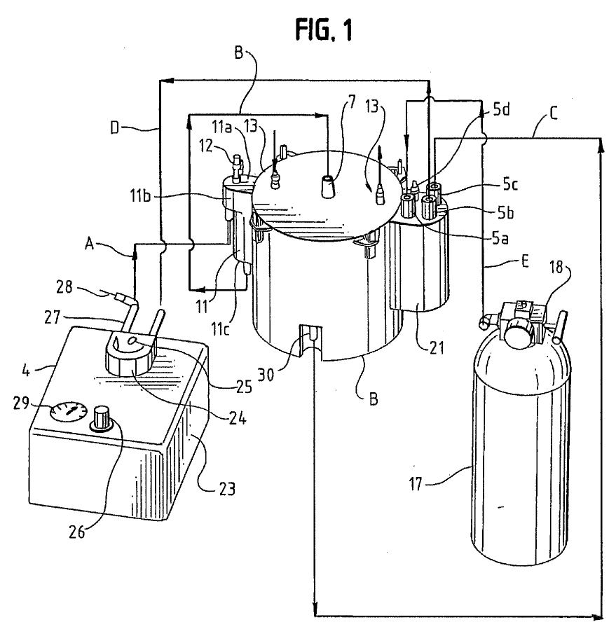

As shown in FIG. 1, one embodiment of the perfusion apparatus of the present invention includes a compressed oxygen canister 17, an oxygenator chamber assembly 21, an organ container 8, an organ container lid 9, a bubble remover 11, a pump assembly 4 and one or more cooling blocks or freezer packs 6.

The oxygen supply 17 is coupled to the oxygenator 21 through a pressure regulator 18. The oxygenator 21 is attached to the side of the reservoir or organ container 8. Similarly, the bubble remover 11 is attached to the organ container 8 thus providing a compact assembly. The function and operation of the oxygenator 21 and the bubble remover 11 will be described in more detail below. The bubble remover can also be independent of the organ container 8 or integrated into the organ container or another part of the apparatus.

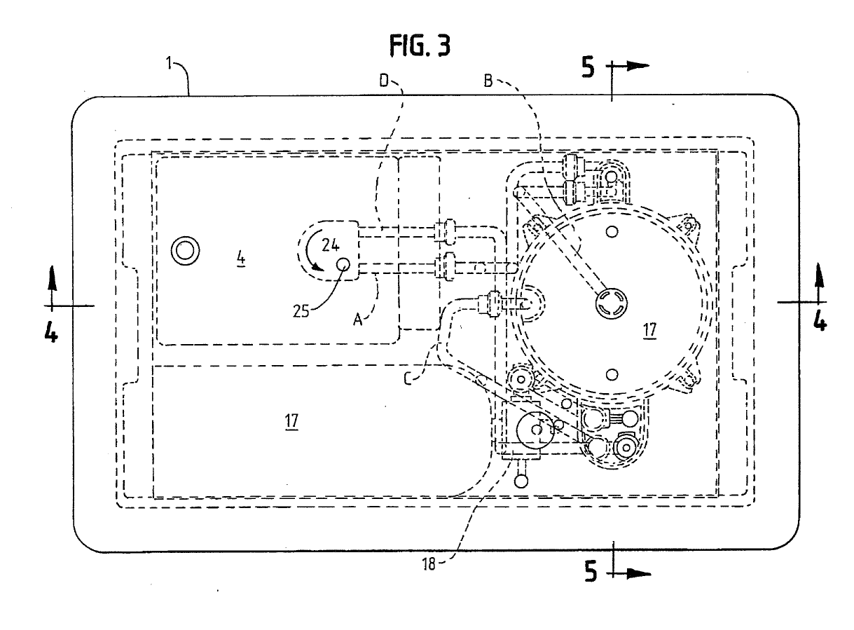

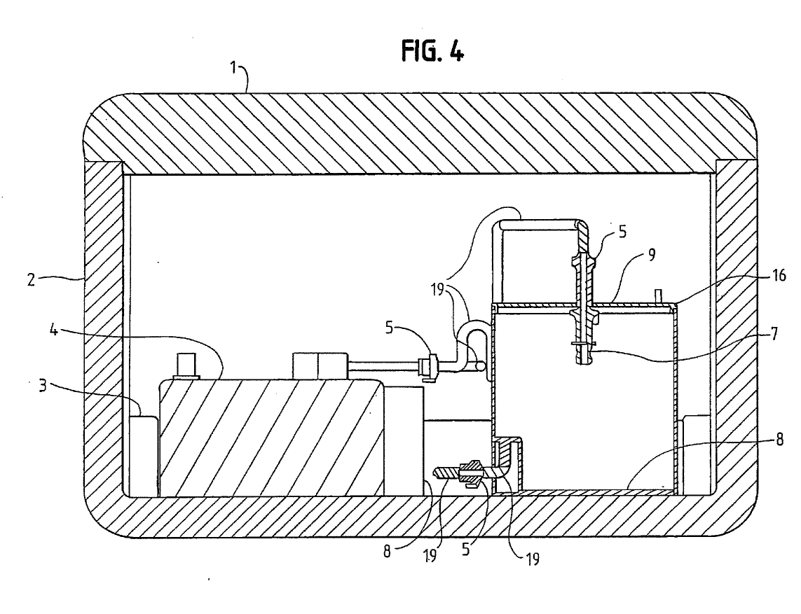

As shown in FIG. 3, the organ container 8 together with the oxygenator assembly 21 and the bubble remover 11 occupy approximately one third of a cooler 2 while the oxygen canister 17 together with the pump assembly 4 and cooling blocks 6 occupy the remainder of the cooler 2. The aforementioned components can be mounted on a tray 3 as shown in FIG. 3. The cooler provides for a compact and readily transportable assembly of approximately 50 quarts (47 liters). The weight of the entire assembly, including the organ to be transported and the perfusion fluid, preferably does not exceed 50 pounds (23 kg).

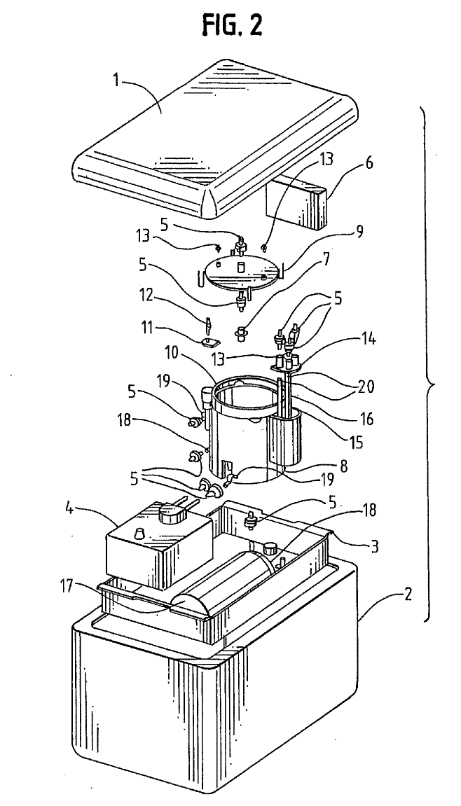

FIG. 2 shows how the main components, the oxygen canister 17, the oxygenator assembly 21, the organ container 8, the bubble remover 11, the pump assembly 4 and cooling blocks 6 fit onto the tray 3 and into the container 2.

The main components can be manufactured by injection molding using a polycarbonate resin suitable for medical use such as Makralon® Rx-1805 or ULTEM®1000. This thermoplastic resin is a transparent polycarbonate formulated to provide increased resistance to chemical attack from intravenous (IV) fluids such as lipid emulsions. Other biocompatible injection molding resins are also contemplated for use herein.

A biocompatible barrier layer can optionally be applied to the fluid contacting walls of the device, as necessary to prevent development of endotoxins due to shedding of particles or the like from surfaces of the components that come into contact with the organ or perfusion fluid. This can easily be accomplished with a number of compounds, for example, medical grade Silastic® organosiloxane elastomer material, available from Dow Corning Corp. This compound comes in many forms including a liquid material that can be painted onto any surface and dried by exposure to air or UV light. Once applied it provides a liquid tight barrier that does not leach, prevents contact at a biochemical level between compounds on either side, and has repeatedly been shown to be biocompatible for long periods, as when used as a part of numerous permanent implants in a number of medical fields.

The use of suitable biocompatible materials for the components contacting perfusion fluid prevents activation of the complement factors of the immune system of the organ by materials of the organ container and other parts to which the perfusion fluid or organ are exposed. Activation of the complement factors of the immune system may occur if the organ is exposed to toxins while in the organ transport unit, and may shorten the amount of time the organ can be transported without losing viability as a transplant. The use of highly biocompatible materials will help keep organs physiologically healthier and potentially provide both healthier organs for transplant and a longer permissible transport time for individual organs being transported.

The perfusion solution can be a complex mix of buffers and small molecular weight molecules that can be pumped through the organ to provide nutrients, maintain its pH and to chemically slow its metabolism. Further, the solution itself provides a medium to chill the organ to the low temperature at which the fluid is maintained. The perfusion fluid contemplated for use in the present invention can be, for example, the fluid sometimes referred to in the literature as “Wisconsin solution.” A commercial source of Wisconsin solution is ViaSpan® solution, commercially available from DuPont Merck Pharmaceutical Company, Wilmington, Del. Wisconsin solution can also be modified by adding a blood thinner such as heparin, antioxidants, cardiac stimulants, and other ingredients.

In one embodiment, the solution also provides scavengers for oxygen (O₃) free radicals, which radicals are believed to interfere with normal cell function. One scavenger contemplated for use herein is Adenosine. Another contemplated scavenger is Vitamin E. Other oxygen free radical scavengers known in the art are also contemplated for use herein. The scavenger can be any scavenger approved for use in cardiac, perfusion, or IV fluids, now or in the future.

The scavenger optionally can be stabilized within the fluid environment. Stabilizing the scavenger within the fluid will keep the scavenger active throughout transport of the organ. The scavenger can be stabilized, for example, by cross-linking it to a larger carrier molecule (such as glutaraldehyde) in a way that exposes the active binding site, allowing binding to O₃. The size and chemical nature of the cross-linked molecule can be such as to prevent the Adenosine or other scavenger from being absorbed and bound by the heart tissue.

Another approach is to provide the scavenger in a fixed position away from the heart but within the flow of the perfusion fluid. The scavenger is fixed to a platform or substrate, which can be located at a distance from the organ. The free oxygen radicals are picked up as the fluid circulates over the platform, thus effectively removing them from contact with the organ. The scavenger can be fixed to a substrate such as the inner wall of the organ container 8 or another structure disposed within the perfusion fluid circuit and exposed to the circulating perfusion fluid. The platform can optionally be a fluid-permeable filter impregnated with the scavenger.

Yet another approach is to provide a time-release device to deliver the scavenger to the system over time, at a constant or varying rate. Such technology already exists for the delivery of hormones, as in an implant made from Silastic® organosiloxane material. In this case the scavenger molecule is imbedded within or dispersed in the implant. Once placed in the organ container 8, the scavenger is released from the silastic at a steady release rate. As the organ picks up and removes the scavenger from the fluid, the implant release as fresh scavenger into the fluid environment, creating a renewed supply and preventing a buildup of damaging free oxygen radicals within the perfusion fluid.

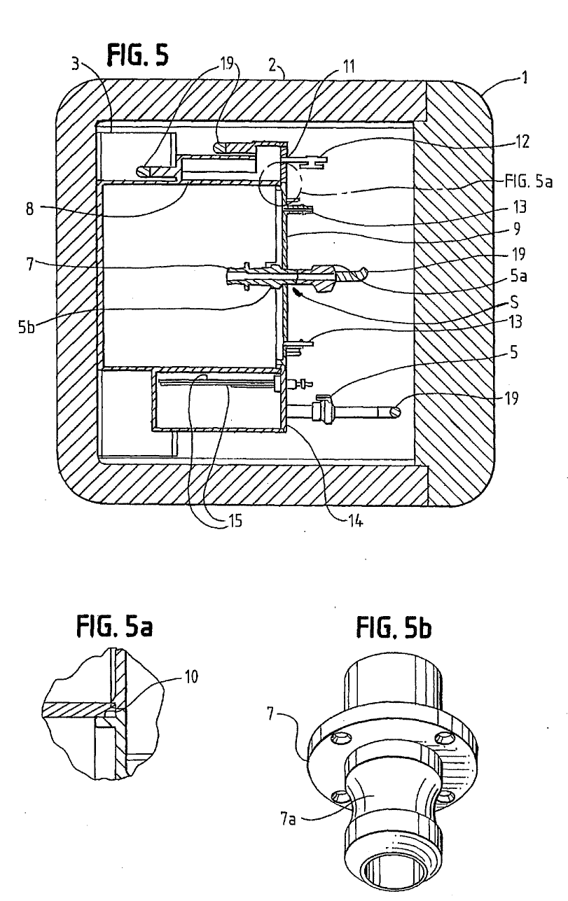

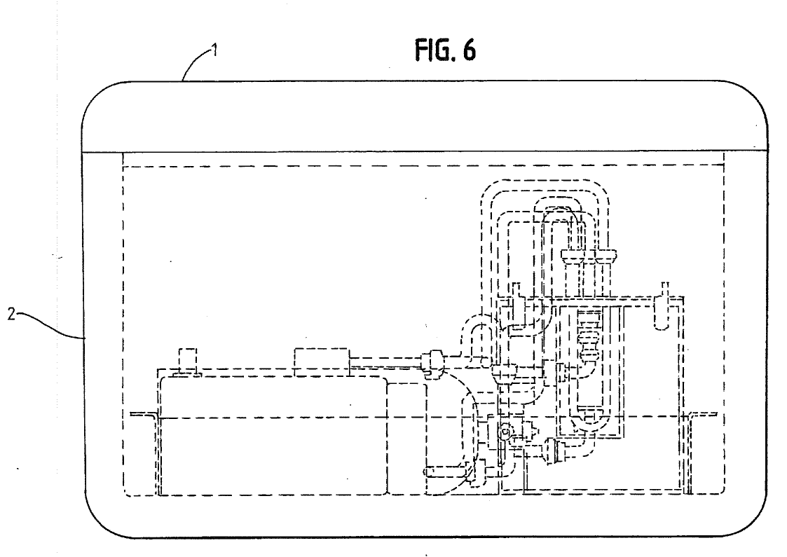

The cover 9 for the organ container 8 can be sealed to the container 8 by a standard O-ring 10 as shown in FIG. 5a or a suitable gasket. Suitable fasteners, adhesives, clamps, straps, latches, or other expedients can be used to hold the cover 9 in place.

The cover 11a for the bubble remover 11 and the cover 14 for the oxygenator 21 can be secured in any suitable manner such as any of the expedients described for the organ container 8. For example, they can be glued in place using a U.V. cure adhesive. The organ and perfusion fluid can be thus sealed from the atmosphere and sterile conditions can be maintained.

The tubing 19 used to connect the various components together can be made from USC class 6, manufactured by many suppliers. Quick connect-disconnect couplings 5 can be used throughout the assembly. One such fitting is manufactured by Colder Products and requires only one hand to operate. The fittings 5 are FDA approved and are readily available.

The assembly of the tubing 19 to the fittings 5 may be accomplished by pushing the tubing 19 onto tapered bosses 22. No barbs on the bosses are necessary due to the low pressure of the system, which can operate at slightly greater than usual atmospheric pressure, such as less than 2 bars absolute. An alternative option is to solvent bond or U.V. bond the tubing 19 to the tapered bosses 22. Since the tubing and the other parts of the perfusion loop are optionally disposable after a single use, there may be no need to disassemble them. Optionally, certain parts of the apparatus, such as some or all disposable elements, can be joined together in advance using tubing welded or glued into place to form connections.

Centrally located on the underside of the organ container cover or lid 9 can be a standpipe or adapter 7. This adapter can be connected to the bottom of cover 9 by a quick disconnect coupling 5. The adapter can be designed so that, for example, in case of a human heart the aorta may be attached to it. Optionally, the adapter 9 and the lid 11 can be integrated into a single part, made in one piece or assembled from more than one piece.

While a generally cylindrical organ container is disclosed, other cross-sections such as oval or rectangular may be used.

The oxygenator 21 can be in the form of a hollow chamber with a cover 14 and can be attached to the organ container 8. The cover 14 can be equipped with 3 quick connect fittings 5a, 5b and 5c and one check valve 13 through which gases may be vented to the atmosphere. The corresponding quick connect fittings 5, the tubing used to connect them, or both can be color coded so that improper connections can be avoided. A quick connect oxygen inlet fitting 5a communicates with the interior of the oygenator 21 by (for example) 4-6 gas permeable Silastic® polymer tubes 22 through which oxygen can be transferred to the perfusion fluid in the oxygenator 21. The flow of oxygen through the tubes can be opposite to the direction in which the perfusion fluid flows through the oxygenator 21. This increases the efficiency of oxygen transfer to the fluid. The tubing is manufactured by Dow-Corning and is sold under catalog number 508-006. In one embodiment, the tubing 22 has an inside diameter of 0.058 inches or 1.47 mm and an outside diameter of 0.077 inches or 1.96 mm. The oxygenator tubes can be 24 inches long. Quick connect fittings 5b and 5c communicate with the interior of the oxygenator 21 and can be used to supply used perfusion fluid for oxygenation through the fitting 5c and withdraw oxygenated perfusion fluid through the fitting 5b. Excess oxygen can be bled to the atmosphere through check valve 5d, to avoid foaming and bubbles in the perfusion fluid.

While an exemplary device uses Silastic® tubing for gas exchange, it should be understood that other silicone tubing or other materials may be used. For example, polyethylene can be permeable to oxygen and carbon dioxide but not aqueous solutions. It is, however, rigid. Thin polyethylene sheets can be used to make a functioning oxygenator in an assembly like an automobile radiator. Such an assembly could, for example, be housed inside a tube which is connected in line with the perfusion fluid path.

The bubble remover 11 can be in the form of a hollow chamber with a lid 11a. The chamber 11 has an upper portion 11b and a lower portion 11c. The cross-sectional area of the upper portion 11b of the chamber 11 can be larger than the cross-sectional area of the lower portion 11c. The lowermost parts of the upper and lower portions of the chamber 11 can be provided with quick connect fittings 5 that communicate with the interior of the chamber 11. The cover 11a of the chamber 11 can be equipped with a one-way venting valve 12 through which gases can be vented to the atmosphere. Alternatively, the bubble trap and vent may be molded and integrated into the top of the organ container 9 such that it is inline with the fluid path.

It will be readily apparent to those skilled in the art that other forms of bubble removers may be used, such as one having a different cross-sectional area.

The pump assembly 24 comprises a sealed rechargeable lead-acid or lithiumion 12-volt battery 31, a DC brush motor 32 and an AC transformer and AC/DC converter 33 to supply 12-volt DC to the motor when AC current is available. The motor shaft drives the pump 24. The pump 24 can be a peristaltic pump manufactured by APT Instruments and has a capacity of 8-10 milliliters/min/100 grams of organ weight. A human heart weighs approximately 400 grams. The pump 24 can be mounted to the outside of the box and the pump on-off switch 25 can be mounted on the pump, thus providing ready access. A pump r.p.m. gauge 26 can be mounted on the outside of the box 23. Pump r.p.m. is an indication of the flow rate of perfusion fluid. A pressure cuff 27 or pressure transducer 28 may be mounted on the fluid supply line A or inside a T-connection in case a pressure transducer is used. A pressure readout gauge 29 can be mounted on the box. Appropriate pressure, temperature and fluid flow alarms (not shown) may be mounted on the box or in another convenient location such as on the cooler 2.

Other forms of pumps may be advantageously used, for example, syringe pumps or centrifugal pumps may be readily substituted for the rotary roller pump (peristaltic pump) disclosed.

The invention is useful for the transport of human organs such as the heart, kidneys, livers, lungs and the pancreas. The operation of the device will be described in connection with a human heart.

When a heart donor becomes available the surgeon removes the heart from the donor in the sterile environment of an operating room.

The tray 3 carrying the organ container 8 and the attached oxygenator 21 and bubble remover 11 together with the pump assembly 4 and oxygen bottle 17 are present to receive the heart, which can be first emptied of blood with perfusion fluid. This is standard procedure. The aorta can then be connected to the concave portion 7a of the adapter 7, as by suturing. The heart can then be suspended in the organ container 8 partially filled with perfusion fluid. The entire container 8 and the oxygenator 21 can be then filled with fluid. The oxygen container 17 can be connected to the oxygenator 21 by the tube E.

The bottom of the organ container 8 has a perfusion fluid outlet 30 that can be connected to the oxygenator inlet 5c by the tube C so that used perfusion fluid can be transported to the oxygenator 21.

The outlet 5b of the oxygenator 21 can be connected to the pump 24 by a tube D so that oxygenated fluid can be pumped from the oxygenator 21 to the pump 24 and by the tube A into the bubble remover 11 where air bubbles and foam rise to the top and can be removed from the fluid. Commonly, most of the bubbles form early during the course of perfusion.

The fluid travels from the bottom of the bubble remover 11 through the opening 31 and the tube B into the adapter 7, to which the aorta has been sutured. The connection of the tube B to the adapter 7 can be the last connection made which assures that there is no air entering the aorta with the perfusion fluid.

The tray 3 can be now placed in the cooler 2 and coolant blocks 6 can be placed in the cooler to maintain the temperature in the cooler at approximately 4° C. to 6° C., or at another desired temperature.

All connections of the tubes A-E can be made with color-coded quick connect-disconnect fittings 5. Only one hand is needed to operate the fittings 5. Alternatively, the tubes may be welded to the respective connection points and installed as a disposable set into the multiuse apparatus.

A heart can be paralyzed just before it is harvested so that the donor heart is not contracting while being perfused. The oxygen requirement of a non-contracting heart cooled to 4° C. can be 1/100 of the oxygen consumed by an actively beating heart at body temperature (37° C.). The two-liter oxygen cylinder can supply 0.125 liters per minute oxygen for more than 34 hours, or over 160% of the amount needed to supply oxygen for a 24-hour period.

The rate of perfusion can be controlled by controlling the r.p.m. of the pump 24. This may be accomplished by a pulse width modulator (PWM), which is a commercially available device.

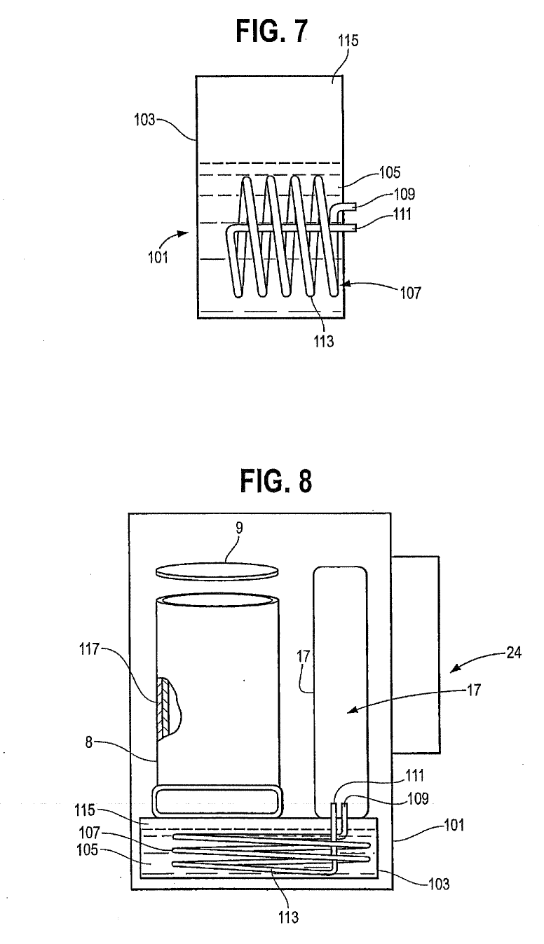

Alternative cooler arrangements are shown in FIGS. 7-9, and may have the advantage of better regulating the temperature at which the organ can be maintained. Referring first to FIG. 7, the cooler arrangement 101 of this embodiment comprises a 10-liter container 103 defined by a relatively thin wall allowing radiant heat transfer, containing about 8 liters of a fluid cooling medium 105 and a cooling coil 107.

The fluid-cooling medium can be, for example, the cooling medium used in commercially available cold packs (for example Polar Pack® coolant, sold by Midlands Chemical Company, Inc., Omaha Nebr.). The coil 107 has an inlet 109 and an outlet 111 projecting through the wall of the container 103 and a central or bight heat transfer portion 113 immersed in the fluid cooling medium 105. A headspace 115 can be provided in the container 103 above the fluid cooling medium 105 to allow for expansion and contraction of the container 103 and the medium 105.

The coil 107 in this embodiment can be made of a one-meter length of stainless steel or other biocompatible tubing, which can be heat-conductive. The tubing can be bent into a convenient configuration, such as a helical coil, and placed into the 10-liter container 103 with the ends of the coil placed through openings made in the container wall. The container can then be filled with the fluid cooling medium 105, preferably taking care to ensure no air pockets are left around or below the heat transfer portion 113 of the cooling coil 107. The container 103 can then be capped and frozen in a conventional freezer at −6° to −17° C. The inlet and outlet 109 and 111 of the cooling coil can be connected by biocompatible flexible tubing to the perfusion fluid circuit, for example by connecting the inlet 109 to the outlet of the bubble remover 11 and the outlet 111 to the adapter 7 as shown in FIG. 1.

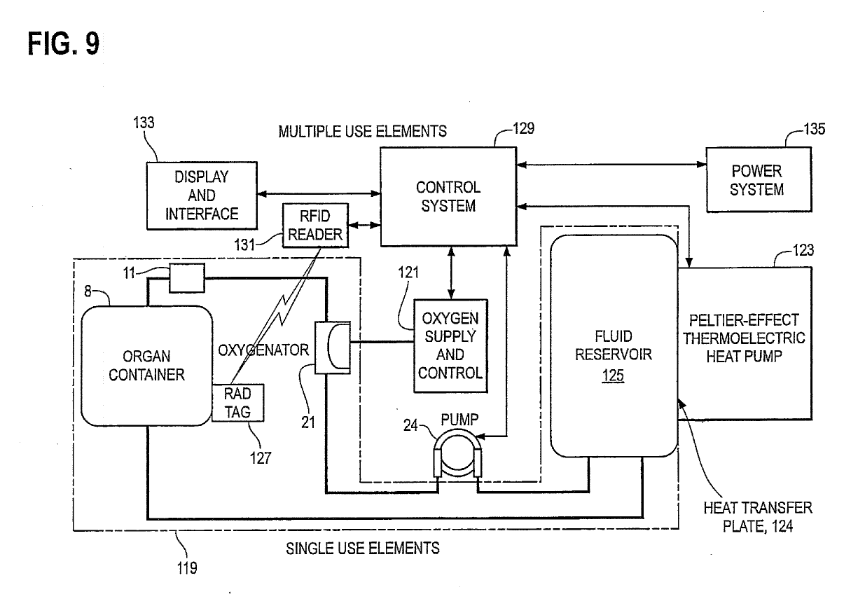

In the disclosed embodiment, this system will cool the entire organ transport unit to 10-13° C. for 24 hrs and the organ container 8 to between 5-8° C. for 12 hrs. However, if the organ container 8 is insulated with a Styrofoam® foamed polystyrene or other insulating material sleeve 117 (as shown in FIG. 9), the fluid in the organ container 8 can be held below 10° C. for well over 24 hrs.

Referring now to FIG. 8, a more compact assembly is shown in which the cooler 101 is located below the organ container 8 and the oxygen bottle 17, which allows the assembly to be more compact.

FIG. 9 is a schematic drawing of an alternative organ transport device that employs a Peltier-effect thermoelectric heat pump. Referring to FIG. 9, the organ container 8, oxygenator 21, oxygen supply and control 121 (including the supply bottle and regulator), and pump 24 can be substantially as previously described.

As shown schematically in FIG. 9, the organ transporter can be provided in the form of a disposable portion 119 and a reusable portion shown in the remainder of the Figure. The disposable portion 119 can include, for example, the perfusion loop components and optionally a tray to support them when they are separated from the reusable part. The tray is not essential, however. The reusable part can include, for example, the outer container, oxygen bottle, battery, chiller, electronics and pump (except for the tubing defining the perfusion path, in certain embodiments).

One advantage of providing one assembly that is disposable after a single use and another reusable assembly can be that the portions of the apparatus requiring sterilization can be limited to those that come in contact with the organ and the perfusion fluid. It is not necessary to sterilize electronics, a battery, the pump impeller, the pump motor, and other parts that can be difficult to sterilize.

The adapter 7, organ container 8, bubble remover 11, oxygenator 21, associated tubing, and a supply of perfusion fluid can be sterilized and provided in the operating room where the organ is harvested, attached to the adapter 7, placed within the organ container 8, and connected by suitable lengths of color-coded disposable sterile tubing to the bubble remover 11, oxygenator 21, and oxygen bottle 17. This assembly is disposable after a single use and forms a closed system isolated from ambient conditions and contaminants.

The closed system can then be removed from the sterile field and assembled with the reusable components of the organ transporter. Electrical connections can be made between the disposable and reusable portions, the organ container 8 can be placed in heat exchange relation to the chiller, and the disposable components can be secured in the outer container to prepare for transport. The process can be reversed at the destination to unload the organ and approach the sterile field for implantation.

Another advantage of a partially disposable and partially reusable assembly can be that many of the expensive components, such as the computer, display, and oxygen bottles, can be reused.

Yet another advantage of a partially disposable and partially reusable assembly can be that the disposable parts can be specially adapted for particular organ types, sizes, and other characteristics, thus multiplying to some degree the different types of disposable parts, while the reusable parts can be adapted to be versatile, for use with any organ type or size or other characteristics. For example, the on-board computer can adapt to the particular associated organ container, as by receiving a signal from its RFID tag 127, to adjust the perfusion fluid temperature, pressure, and flow rate, the oxygen pressure and flow rate, and other conditions to suit the particular organ to be transported. Thus, a single type of reusable assembly may be provided for many or all organ types to be transported. This minimizes the amount of reusable equipment that needs to be purchased, tracked, maintained, and stored in connection with an organ transportation system.

The RFID tag 127 is secured to the organ container 8, preferably in such a way that they cannot become separated. For example, it may be attached by adhesive or held in place by an overlying sheet or sleeve of plastic or other suitable material.

The RFID 127 can be configured (as by initial programming or by programming it at the time of use) to communicate the type of organ the apparatus is designed to carry, labeled to carry, or carrying, and to communicate a serial number for tracking the organ and uniquely identifying it in an Instrument event log. The RFID can also have legible indicia indicating some or all of the same information, so the correct RFID and associated organ container will be used.

A conventional RFID is a passive transmitter; it utilizes the energy content of a signal received from the RFID reader to power its transmitter. A powered transmitter may also be used, however. The power can either be provided by a dedicated battery or transmitted by a connection made with the main battery of the apparatus when the organ transporter is assembled. An RFID reader can be incorporated into the computer control portion of the organ transporter. The organ transporter software can react to the RFID transmission by automatically configuring the organ transporter to suit the container (size and/or organ type) and to create a uniquely identified log file from the serial number transmitted by the RFID.

Using an RFID to automatically configure the organ transporter and sensors to determine the state of the transporter and its transported organ has the advantage that relevant parameters such as the perfusion pressure or flow rate, steady state temperature, temperature profiles, oxygen pressure, nutrient levels, metabolite levels, maximum transport time allowed, or other parameters which may vary by organ type or size or the manner in which the organ was harvested (for example, an organ from a recently-deceased donor might require different handling than an organ from a brain-dead, heart-alive donor) can be measured or calculated and properly maintained, without the need for the transporter or other personnel to select and implement appropriate parameters. This may reduce the error rate, keep the transported organ viable longer, or make the organ more viable at the time it is delivered.

The embodiment of FIG. 9 has a control system 129, here a microprocessor based digital control system, though a hardware-implemented or analog system can also be used. The control system 129 is operatively connected to a RFID reader 131 (to read the RFID tag 127), and a display and interface 133. The display and interface 133 can be a touch screen, which combines a display and interface, or a conventional screen with push buttons disposed adjacent the screen to provide permanent or changeable indicia for the push buttons (much like some automated bank teller machines presently operate), in which case the push buttons are the interface and an LCD or other display is separate. The display can be any type of display, for example an analog or digital gauge or numerical readout or an LCD display. The term “display” should be broadly construed to include a visible or audible indicator, such as a talking display or alarm. The interface can be any type of interface, for example a mouse, trackball, touchpad, joystick, keyboard, microphone, infrared transmitter (like a remote control), etc. The apparatus shown in FIG. 9 is driven by a power system 135, supplying required DC voltages to the display and control elements.

The arrangement of FIG. 9 further includes a Peltier-effect heat pump 123 thermally linked, as by a common, heat conductive wall 124, to a reservoir 125. Examples of patents disclosing Peltier-effect heat pumps such as 123 are U.S. Pat. Nos. 6,548,750 and 6,490,870, which are hereby incorporated by reference in their entireties. Such a chiller does not require a fluid refrigerant or heat sink; it can be a solid-state device, and can function with no moving parts. The heat pump can interface to a separate fluid reservoir (see FIG. 9) or a co-located fluid reservoir and organ container.

While the Peltier-effect heat pump consumes electricity to pump heat, it has some advantages in the present application. One advantage can be that it needs no refrigerant or coolant and no accompanying apparatus (such as a compressor, evaporator, and condenser, as in a conventional compression refrigeration system), and thus saves weight, which can compensate at least in part for the additional battery capacity required to operate it.

A second advantage of the Peltier heat pump can be that it can be made part of the reusable portion of the organ transporter. The organ container or a separate fluid reservoir can include a high surface-area heat transfer surface, such as a heat-conductive wall or bottom. This heat transfer surface can be part of the unit that is disposable after a single use. This container can be placed in with its heat-conductive bottom or other wall in close thermal conductive contact with a heat-conductive outer surface of a Peltier-effect heat pump. The heat pump mechanism can be part of the reusable portion of the unit. Cooling the contacting surface of the heat pump will cool the vessel and its perfusion fluid content, without the need for circulating the fluid through the heat pump or a conventional heat exchanger.

The thermal contact between the organ container and the heat pump can be improved by placing a liquid, heat-conductive material, such as an aqueous gel, between the heat pump and organ container surfaces when they are mated. If the heat-conductive surface of the heat pump is dished to nest with a congruent surface of the organ container, the liquid heat-conductive material can be contained so it will not tend to leak out. Alternatively or in addition, the liquid heat-conductive material can be liquid as applied, then fuse, cure, or otherwise harden or become viscous to form a heat-conductive solid interface between the organ container and the heat pump.

Another contemplated alternative can be to use the heat-conductive wall of the organ container as one component of the Peltier heat pump cooling element. This avoids the need to provide a separate wall and cooling element, and may improve the heat transfer rate between the organ container and the cooling element.

A third advantage of the Peltier heat pump can be that it can be used to either heat or cool the perfusion fluid, merely by reversing the flow of electricity in the Peltier-effect heat pump. If the transporter is being carried in a very cold environment or used to re-warm the organ near the end of transport, it can heat the perfusion fluid.

Moreover, the Peltier heat pump can be maintained at any given temperature; it is not limited to an inherent temperature. This property is in contrast to ice or another cooling medium that cools its environment as it melts, maintaining a temperature closely approaching its melting temperature.

The ideal temperature at which an organ should be held to maintain it over a long period is still being investigated, but there are indications that the ideal temperature should be maintained within a narrow range, and the best temperature may be substantially higher than zero degrees Celsius. Some contemplated temperatures can be in the range from greater than 0° C. to 12° C. Some particularly contemplated minimum temperatures for the organ can be 1° C., alternatively 2° C., alternatively 3° C., alternatively 4° C., alternatively 5° C., alternatively 6° C., alternatively 7° C., alternatively 8° C., alternatively 9° C., alternatively 10° C., alternatively 11° C. Some particularly contemplated maximum temperatures for the organ can be 12° C., alternatively 11° C., alternatively 10° C., alternatively 9° C., alternatively 8° C., alternatively 7° C., alternatively 6° C., alternatively 5° C., alternatively 4° C., alternatively 3° C., alternatively 2° C., alternatively 1° C. Any stated minimum temperature can be associated with any stated maximum temperature that is as great or greater to define a specifically contemplated temperature range.

Still another advantage of this embodiment can be that the Peltier chiller can be used to provide a heating or cooling profile for the organ. The organ normally will be harvested at a temperature between ambient temperature and normal body temperature, cooled to a transport temperature, and either preheated before being implanted or reheated to body temperature by the recipient's metabolism as the organ is implanted and starts to function.

While the appropriate temperature profile is under study at present, it is contemplated that the organ can be placed in the organ transporter, cooled at a desired rate or following a desired temperature-time profile, transported, and then heated at a desired rate or following a desired temperature-time profile, after which it can be transplanted into the recipient. Cooling and re-heating the organ in the transporter as it is being transported can save some of the time between harvesting the organ from the donor and transplanting the organ into the recipient. This is contemplated to be particularly useful, and may extend the transportation time the organ can withstand successfully and still be transplantable, if the desired heating or cooling cycle requires a substantial time to complete.

Many other improvements to the present invention are contemplated, for example, the following.

In place of a mechanical oxygen pressure regulator that can be manually adjusted, a computer-controlled regulator can be used that allows variable pressure or flow control based on an integrated downstream fluid oxygen partial pressure sensor. The computer controlled regulator can be used to adapt the oxygen partial pressure or flow rate provided to suit organs of different sizes and types, such as juvenile versus adult organs, or hearts versus kidneys, based on inputted data indicating the size and type of organ being transported. The transporter can automatically adjust in a variety of ways to the size and type of organ being carried, based on elementary entries by the operator (or RFID tag) indicating the size and type of organ. It can also adjust to changes in oxygen consumption baked on organ metabolism over the life of transport.

In an embodiment of the invention, additional adaptations may be made to purge or prime the perfusion fluid loop in the organ transporter. In place of a manually controlled venting valve or check valve 13 (see e.g. FIG. 1) to vent gas from the fluid loop of the system for purging or priming, an electro-mechanical solenoid valve 13 can be provided, which can be computer controlled (or optionally can also be manually controlled). An ultrasonic, electrical conductivity, or thermal conductivity liquid/air detector can be incorporated in the fluid loop, so the computer can control automatic priming and air purge using the solenoid valve to vent gas when necessary at any time when the transporter is in use.

For example, a detector can be placed near the valve 13 in the adjacent headspace (which is broadly defined here as the area normally defining a headspace, whether or not it contains a gas at a given time) that can detect whether there is gas or liquid in the headspace. The processor can be programmed to vent the headspace whenever excess gas requiring venting is present, or it can be programmed to keep the valve closed if there is insufficient gas in the headspace or no gas requiring venting.

The same detector or a separate detector can also be adapted to detect whether the pressure in the headspace is greater than or less than atmospheric pressure. The processor can then lock the valve 13 to prevent it from opening at any time when the pressure in the headspace is less than ambient pressure (ambient pressure can also be sensed by a detector exposed to ambient pressure), or when the pressure in the headspace is so close to ambient pressure that it is not clear which is greater (which might dispense with the need for an ambient pressure sensor, or provide a failsafe function in case one of the pressure sensors is malfunctioning or not properly calibrated). This pressure sensing function may not be necessary while the organ container is in a sterile field, and might even be ignored at that time so a visual indication that the purging is complete is provided by fluid visibly exiting the valve, but it can be particularly important after the organ container is removed from the sterile field, as for transport.

Alternatively, the difference between the pressure inside the headspace and that outside the headspace can be measured directly by providing a diaphragm in the perfusion fluid loop separating a region within the perfusion fluid loop from a region outside the perfusion fluid loop. Deflection of the diaphragm toward one region or the other or a force imposed on the diaphragm can be measured to determine the magnitude and direction of the pressure differential between the headspace and the ambient condition.

Alternatively, the valve 13 can be a check valve that only permits flow out of the perfusion fluid loop, and only opens to permit such flow when the pressure presented at the inlet of the valve 13 is greater than the pressure at the valve outlet.

This processor-controlled venting of headspaces can be used to purge gas from the perfusion fluid loop when the perfusion fluid is loaded into the organ transporter to prepare for use. If the detector detects only gas in the headspace and the pressure within the headspace is greater than ambient pressure, as when the fluid loop is being filled (and the entering fluid compresses air or other gas within the fluid loop), the valve 13 can be opened to vent the headspace.

The embodiment shown in FIG. 1 has a fixed opening in the fluid path, which means that the resistance of the fluid path to flow of fluid is fixed. This is so because the components of the fluid path have essentially fixed flow cross-sections and lengths. Therefore, in the embodiment of FIG. 1 the fluid pressure can be controlled mostly by the pump flow rate against organ resistance.

The pressure sensor of the organ transporter can be used to sense the perfusion pressure. In this embodiment, the optimal perfusion pressure can be calculated by the computer based on the type of organ and its mass. The actual perfusion pressure can be varied to approach or achieve the optimal rate.

In this embodiment, the optimal perfusion pump r.p.m. (and corresponding flow rate) can be calculated by the computer based on the type of organ and the mass. The actual flow rate can be varied to approach or achieve the optimal rate by regulating the volumetric pumping rate of the pump 24. The perfusion flow rate can also be sensed (or determined from the pump rotation rate or the current drawn by the pump or the voltage drop across the pump, depending on the type of pump employed) and computer controlled to allow any flow rate within predefined ranges. In this embodiment, the perfusion fluid pump 24 can be a variable-rate pump. For example, if the pump 24 is a peristaltic pump, the rate of travel of its impeller can be varied to vary the volumetric pumping rate.

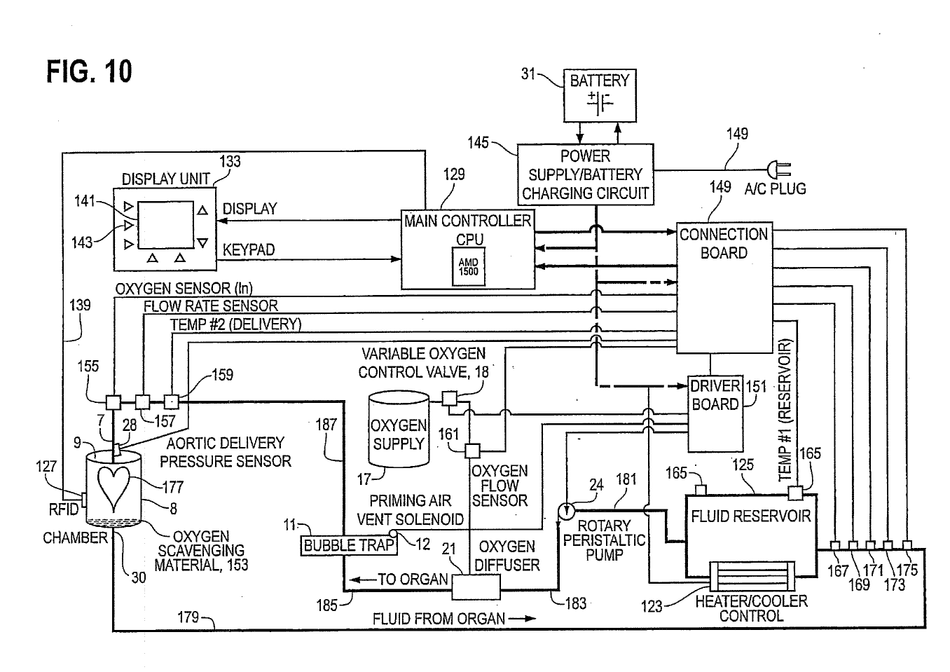

FIG. 10 shows a more developed electronic control system and perfusion fluid loop for an organ transporter 137 according to another embodiment of the present invention. In addition to the components previously described, the transporter 137 of FIG. 10 also includes a display screen 141, control push buttons such as 143, an integrated power supply and battery charging circuit 145 with a/c power cord 147, an electronic interconnect connection board 149, a driver circuit board 151, oxygen scavenger material 153 to remove free radicals, and an array of sensors. The sensors can include, for example, a pressure transducer 28, an oxygen sensor 155, a flow rate or pressure sensor 157, a delivery temperature sensor 159, an oxygen flow sensor 161, a reservoir pressure sensor 163, a reservoir temperature sensor 165, and organ fluid output sensors 167 (generally), 169 (one or more metabolite sensors), 171 (potassium), 173 (sodium), and 175 (oxygen concentration). These sensors are exemplary, and more or fewer sensors or different sensors may be appropriate in a given situation or device.

Referring now to FIG. 10, the fluid flow through the fluid circuit proceeds as follows. Perfusion fluid is injected into the organ 177, here depicted as a heart. The aorta of the heart is sutured to the adapter 7, which directs perfusion fluid through the vascular bed of the heart 177. Perfusion fluid leaves the heart 177 through open vessels and is collected in the organ container 8. The oxygen radicals remaining in the draining perfusion fluid are trapped in the oxygen scavenging material 153, after which the perfusion fluid leaves the outlet generally indicated at 30 of the organ container 8. The perfusion fluid drains through the drain line 179 to the reservoir 125, and contacts the reservoir and drain line sensors 163-175 which sense the condition of the perfusion fluid, as by sensing its temperature and pressure, the quality and quantity of metabolites, potassium concentration, sodium concentration, and oxygen concentration of the perfusion fluid. Optionally, further apparatus can be provided to remove metabolites, reestablish desired levels of potassium and sodium, add nutrients, measure carbon dioxide levels or other blood gases, etc. The temperature of the fluid is modified as needed by the Peltier-effect heat pump 123 to either maintain the temperature at the sensor 165 constant or provide a suitable temperature profile.

The fluid in the reservoir 125 then is pumped by the pump 24, operation of which is controlled by the CPU 129 via the driver board 151 and connection board 149, through the reservoir drain line 181 and the oxygen diffuser input line 183, to the oxygen diffuser 21. The oxygen diffuser can add a variable amount of oxygen to the perfusion fluid, depending on the oxygen sensed in the fluid by the oxygen sensor 175, alternatively supplemented or replaced by a determination based on other data from which the amount of oxygen required can be calculated. The amount of oxygen added is controlled by regulating the flow rate or pressure of oxygen delivered through the valve 18, as determined by the flow sensor 161. The flow of oxygen can be increased if the perfusion fluid is substantially depleted, or reduced if the perfusion fluid is less depleted, or the flow rate of perfusion fluid through the diffuser 21 can be increased or decreased to decrease or increase the average contact time between the oxygen and fluid, the pressure of the oxygen can be regulated to control the rate of transfer to the perfusion fluid, or other expedients can be used to regulate the introduction of oxygen into the perfusion fluid at the diffuser 21.

Upon leaving the diffuser 21 through the drain line 185, the oxygenated perfusion fluid is passed to the bubble trap 11, where gaseous constituents are separated from the liquid perfusion fluid, such as by gravity, and the gaseous constituents rising to the top of the trap 11 are expelled through the priming air vent solenoid 12. The de-gassed perfusion fluid then leaves the bubble trap 11 via the organ input line 187.

The organ input line 187 brings the perfusion fluid into contact with an oxygen sensor 155, flow rate or pressure sensor 157, temperature sensor 159, and optionally other sensors as described above (or other sensors not described above), which sense and feed back the condition of the perfusion fluid as it is passed via the adapter 7 back into the organ 177. Deviation from the ideal values sensed at the sensors 155-159 can be fed back to the CPU 129 via the connection board 149. The CPU can then transmit an alarm or react to the deviant conditions to restore the proper composition and condition of the perfusion fluid passed into the adapter 7. As one example, the output of the sensor 157, which senses the back pressure fed to the organ 177, can be fed back to regulate the rate of impeller rotation, and thus the flow rate, at the pump 4 to maintain a constant pressure at the sensor 157. Other expedients can also be made to regulate the pressure, as by controlling the operation of the vent valve 12 according to the pressure sensed at the sensor 157. A release of gas in the headspace of the bubble trap 1 will also relieve the pressure on the fluid adjacent the headspace.

The sensed condition of the perfusion fluid is transmitted via the connection board 149, and from there to the CPU 129, and from there, as desired, to the display unit 133 which can display predetermined or requested values of relevant parameters, or resulting information (like the oxygen level is too low, for example) on the display screen 141. If corrective action is to be chosen manually, an operator can do so by manipulating the push buttons 143 keyed to information displayed on the unit 133.

The power supply illustrated in FIG. 10 includes a rechargeable battery 31 operatively connected to all of the electric power consuming parts of the assembly. A power supply and battery charging circuit 145 is also provided to accept household or institutional alternating current power and use it to charge the battery 31 and/or power the other components of the system. Single-use batteries can alternatively be used to power the transporter.

Referring still to FIG. 10, the organ transporter 137 can optionally include an interactive user interface including a color display 141 and data entry pad including keys such as 143, which can be associated with elements of the visual display 141 or bear suitable icons or alphanumeric characters for data entry. The data entry pad can include software-programmable membrane key switches such as 143 or other types of keys. Other types of data entry devices, such as a mouse, touchpad or other pointing device, voice recognition software, or others, can also be provided. Using the interface, an operator can enter the mass and weight of the organ, the type of organ, the blood type, age, weight, or other characteristics of the donor, and other pertinent data. Any or all of the parameters mentioned above with respect to the RFID might be entered or changed, for example.

The color display 141 of the interactive user interface 133 can provide the minimum value, the maximum value, and continuous current value updates for all monitored parameters and metabolites sampled from the organ and/or the perfusion solution. This will help in viability assessment at the receiving end of transport.

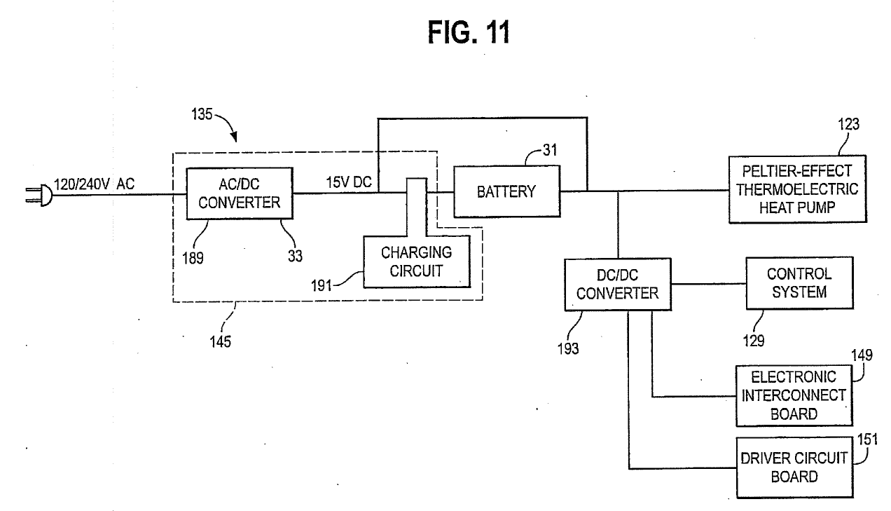

FIG. 11 shows more details of an AC/DC power supply circuit 135 for the organ transporter 137 shown in other Figures. The AC/DC converter 189 can include a power transformer to reduce the AC voltage, a rectifier to convert AC to pulsating DC, a filter capacitor to provide constant-voltage DC, or other circuit elements to convert the household or institutional 120 or 240-volt feed to DC having an appropriate average and instantaneous voltage to operate the charging circuit 191. For example, the current fed to the charging circuit 191 can be nominally about 15 volts DC, to fully charge the battery 31 even though the nominal voltage will drop under load and as the result of powering the charging circuit 191. The battery charging circuit 191 is connected to the battery 31, which can be made of one or more rechargeable cells. The AC power can be selectively directed from the AC power source 145, the battery 31, or both in parallel to the electrical and electronic components of the organ transporter 137. A rechargeable battery 31 can be replenished by connecting AC power, and can be permanently or durably mounted in the reusable portion of the device, so there is no need to provide an access door or other provisions for replacing the battery. AC power can also be used to replenish the battery while an organ is in transport, as when the transporter is waiting in an airport for the next scheduled flight. A readout of the battery charge remaining can also be provided.

In this embodiment, the DC voltage drawn from the battery 31 is supplied at one DC voltage to the heat pump 123 and at another DC voltage to the control system 129, connection board 149, and driver board 151. One expedient to supply two different DC voltages from a single battery is to provide a DC/DC converter 193, so the heat pump 123 is provided directly with current at full battery voltage, while the control system and other components are provided with current at a lower battery voltage suited for their operation. The specific components that must be operated at one voltage versus another will vary depending on the equipment and conditions selected. Another consideration leading to the use of two different DC outputs is that the voltage supplied to the heat pump 123 will vary depending on the amount of cooling or heating desired, and the polarity of the voltage must be reversed to switch from heating to cooling or vice versa. These factors make it desirable to have different DC power sources for the heat pump 123 and other components, which are electronic and typically will be operated at a substantially uniform DC voltage and an unchanging polarity. Of course, more than one battery 31 having different voltages could also be provided, and the charging circuit 191 could accommodate both of them, in another embodiment of the invention.

The pump assembly 24 shown in FIG. 1 has two quick disconnects such as 27 at the fluid path inlet and outlet of the pump 24. As a result, the tubing or other fluid-receiving portion of the pump assembly 24 between its fluid path inlet and outlet must be cleaned or replaced to reuse the organ transporter.

In an alternative arrangement for the pump 24, the single-use disposable tubing already used to plumb the pump 24 into the perfusion loop can be the pump element flexed by the impeller to pump the perfusion fluid.

Referring to FIG. 1, the pump 24 can be a peristaltic pump and the tubing defining the fluid input and output can be an unbroken length of flexible tubing connected at one end to the quick connect fitting defining the outlet 5b of the oxygenator 21 (FIG. 1), and at the other end to the quick connect fitting defining the inlet of the bubble remover 11 (FIG. 1). A bight or intermediate portion of the tubing can be laid along the path traversed by the impeller of the peristaltic pump 24.

A person with ordinary skill can readily obtain a tube loadable pump 24, which is commercially available. In a preferred embodiment of the invention, as described above, the pump 24 can be adapted to facilitate one-handed loading of a bight portion of tubing in the pump assembly 24 that is disposable after a single use. One contemplated tube loadable pump is a linear pump having a straight reaction block and a linearly traveling impeller, so the tube can be easily loaded by placing a straight run of tubing in the impeller and reaction block assembly.

It will thus be seen that we have provided a portable organ transport device that will maintain the viability of an organ for 24 hours or more. The device can be compact in construction and light in weight.

The entire assembly can be housed in a commercial cooler holding approximately 50 quarts (47 liters), or alternatively a similarly insulated rigid container, and the total weight can be less than 75 pounds (34 kg), optionally less than 60 pounds (27 kg), optionally approximately 50 pounds (23 kg) or less.

The many benefits of our invention include the ability to deliver organs in better physiological condition, to shorten recovery times, to reduce overall cost, to increase the available time to improve tissue matching and sizing of the organ, to perform clinical chemistries and diagnostic testing for infectious diseases prior to transplantation, to enlarge selection of donor organs, to widen the range of available organs, to provide surgical teams with more predictable scheduling and relieving transplant centers of crisis management. Finally, the invention makes feasible a worldwide network of donors and recipients.

What is claimed is:

1. Portable apparatus for maintaining an ex vivo organ in a viable condition for transplantation, the apparatus comprising:
A. an organ container comprising an interior space for receiving an organ to be transported, an opening for passing an organ to be transported, and a lid for closing the opening;
B. a bubble remover comprising a headspace and a venting valve;
C. an oxygenator comprising a chamber for receiving perfusion fluid, a gas space for receiving oxygen, and a gas exchange interface allowing gas exchange between the chamber and the gas space;
D. a perfusion loop comprising the organ container interior space, the bubble remover headspace, and the oxygenator chamber interconnected to provide fluid circulation, in which the perfusion loop further comprises a heat exchange surface and a flexible tube, in which the heat exchange surface of the perfusion loop is at least a portion of the organ container;
E. a chiller configured for operative association with the heat exchange surface to cool a perfusion fluid circulating in the perfusion loop, in which the electric chiller is in heat-exchange contact with the heat exchange surface;
F. a reservoir, wherein a wall of the reservoir defines the heat exchange surface;
G. in which the organ container, the bubble remover, the oxygenator, the reservoir, the heat exchange surface, and the perfusion loop are permanently mechanically joined together in fluid-conducting relation to define a single, sterile, closed unit enabled to be moved as a unit; and
H. in which the single, sterile, closed unit is movable into and out of an operative relationship with a perfusion pump while the perfusion loop remains closed.

2. Portable apparatus for maintaining an ex vivo organ in a viable condition for transplantation, the apparatus comprising:
A. an organ container comprising an interior space for receiving an organ to be transported, an opening for passing an organ to be transported, and a lid for closing the opening;
B. a bubble remover comprising a headspace and a venting valve;
C. an oxygenator comprising a chamber for receiving perfusion fluid, a gas space for receiving oxygen, and a gas exchange interface allowing gas exchange between the chamber and the gas space; and
D. a perfusion loop comprising the organ container interior space, the bubble remover headspace, and the oxygenator chamber interconnected to provide fluid circulation;
E. in which the organ container, the bubble remover, the oxygenator, and the perfusion loop are permanently joined together in fluid-conducting relation to define a single, sterile, closed unit; and
F. in which the single unit is movable into and out of an operative relationship with a perfusion pump while the perfusion loop remains closed.

3. The apparatus of claim 2, in which the perfusion loop further comprises a flexible tube.

4. The apparatus of claim 2, in which the perfusion loop further comprises a heat exchange surface.

5. The apparatus of claim 4, further comprising a chiller configured for operative association with the heat exchange surface to cool a perfusion fluid circulating in the perfusion loop.

6. The apparatus of claim 4 in which the chiller is a Peltier-effect thermoelectric heat pump.

7. The apparatus of claim 6, in which the heat pump is adapted to selectively heat or cool the perfusion fluid.

8. The apparatus of claim 4, further comprising a temperature control for controlling the temperature of a perfusion fluid in the perfusion fluid loop.

9. The apparatus of claim 8, in which the temperature control is programmed to cool perfusion fluid in the perfusion fluid loop following a specified temperature-time profile.

10. The apparatus of claim 9, in which the temperature control is further programmed to heat perfusion fluid in the perfusion fluid loop following a specified temperature-time profile, after cooling perfusion fluid in the perfusion fluid loop following a specified temperature-time profile.

11. The apparatus of claim 5, in which the heat exchange surface of the perfusion loop is at least a portion of the organ container and the electric chiller is in heat-exchange contact with the heat exchange surface.

12. The apparatus of claim 4, wherein the perfusion fluid loop further comprises a reservoir.

13. The apparatus of claim 12, wherein a wall of the reservoir defines the heat exchange surface.

14. The apparatus of claim 2, further comprising a processor programmed for processing data associated with the apparatus.

15. The apparatus of claim 14, further comprising an input device for communicating to the processor the size and type of organ being transported in the apparatus.

16. The apparatus of claim 14, in which the processor is programmed to adapt a parameter to suit the type and size of organ entered at the input device.

17. The apparatus of claim 16, in which the parameter is oxygen partial pressure or oxygen flow rate.

18. The apparatus of claim 2, further comprising a processor, in which the venting valve of the bubble remover is controlled at least in part by control signals from the processor.

19. The apparatus of claim 18, further comprising a gas sensor for detecting the presence of gas in the headspace requiring purging, the processor being programmed to open the venting valve to vent gas when the gas sensor detects the presence of gas in the headspace requiring purging.

20. The apparatus of claim 19, further comprising a gas sensor for detecting the absence of gas in the headspace requiring purging, the processor being programmed to close the venting valve when the gas sensor detects the absence of gas in the headspace requiring purging.

21. The apparatus of claim 19, further comprising a pressure sensor for detecting pressure within the perfusion fluid loop and transmitting data reflecting the pressure to the processor.

22. The apparatus of claim 2, in which the organ container, the bubble remover, and the oxygenator are disposable after a single use.

23. The apparatus of claim 22, further comprising a flexible tube that is disposable after a single use joining at least two of the organ container, the bubble remover, and the oxygenator.

24. The apparatus of claim 23, further comprising a reusable impeller engageable with the flexible tube for propelling perfusion fluid through the flexible tube.

25. The apparatus of claim 22, comprising a portion defining the perfusion fluid loop that is disposable after a single use and a reusable portion not normally exposed to a perfusion fluid in the perfusion fluid loop.

26. The apparatus of claim 2, in which the organ container is disposable after a single use.

27. The apparatus of claim 2, further comprising a radio frequency identification tag installed in fixed relation to the organ container and configured to communicate at least one datum respecting at least one of the organ container and its contents.

28. The apparatus of claim 27, further comprising a radio frequency identification tag reader for detecting data transmitted by the radio frequency identification tag.

29. The apparatus of claim 28, further comprising a processor programmed for receiving data from the reader and controlling the apparatus responsive to the data.

30. The apparatus of claim 29, in which the data represents a parameter selected from at least one of perfusion fluid pressure, perfusion fluid flow rate, perfusion fluid temperature, perfusion fluid temperature-time profile, perfusion fluid oxygen pressure, perfusion fluid carbon dioxide pressure, perfusion fluid nutrient level, perfusion fluid metabolite level, or the maximum remaining transport time allowed for the organ.

31. The apparatus of claim 2, in which the organ container comprises a cover having an inside portion and an outside portion, the apparatus further comprising an adapter having a first portion defining a perfusion fluid inlet, a second portion adapted for connection to a vessel of an organ in the organ container for directing perfusion fluid into the vessel, and a quick connect-disconnect coupling for connecting the adapter to the inside portion of the cover.

32. The apparatus of claim 2, in which the bubble remover is disposable after a single use.

33. The apparatus of claim 2, in which the oxygenator is disposable after a single use.

34. The apparatus of claim 2, in which the organ container, bubble remover, and oxygenator are mechanically joined, enabling them to move as a unit.

35. The apparatus of claim 2, further comprising a support on which the perfusion loop and its components are carried together.

36. The apparatus of claim 5, further comprising a coolant vessel configured to contain a coolant cooled by the chiller, wherein said heat exchange surface is disposed within the coolant vessel for contacting a coolant in the vessel.
